(19) United States
(12) Patent Application Publication
(10) Pub. No.: US 2017/0157529 A1
(43) Pub. Date: Jun. 8, 2017

(54) OIL SEPARATION SYSTEM

(71) Applicant: Fritz Chess, Seattle, WA (US)

(72) Inventor: Fritz Chess, Seattle, WA (US)

(21) Appl. No.: 15/435,858

(22) Filed: Feb. 17, 2017

Related U.S. Application Data
(63) Continuation-in-part of application No. 15/212,342, filed on Jul. 18, 2016.
(60) Provisional application No. 62/315,410, filed on Mar. 30, 2016.
(60) Provisional application No. 62/193,118, filed on Jul. 16, 2015.

(51) Int. Cl. B01D 11/02; C07C 7/10; C11B 1/10

(52) U.S. Cl. CPC B01D 11/0203; B01D 11/0288; C11B 1/104; C07C 7/10

(57) ABSTRACT

A CO₂ extraction system which is capable of being used for extracting compounds, such as oils, from various materials including plant material and botanicals. The extraction system includes a means for supplying CO₂ to a pressure vessel. The pressure vessel is substantially comprised of two distinct portions; a top portion and a bottom portion. The top portion includes a separation zone, while the bottom portion contains a collection zone. Affixed to the bottom portion of the pressure vessel is a drain valve which is capable of allowing extracted material to exit the system. When introducing CO₂ into the extraction system, the CO₂ may be liquefied and further saturated with additional compounds. By maintaining each of the respective top and bottom portions of the pressure vessel at differing temperatures and/or pressures, the various additional compounds may be separated from the CO₂.

This application is a continuation-in-part of U.S. patent application Ser. No. 15/212,342, filed Jul. 18, 2016, which claims priority to U.S. Provisional Patent Application No. 62/315,410, filed Mar. 30, 2016, and U.S. Provisional Patent Application No. 62/193,118, filed Jul. 16, 2015, the contents of which are incorporated herein by reference.

### TECHNICAL FIELD

Provided is an apparatus for separating a dissolved extract from a solvent fluid. More particularly, the disclosed separation method and apparatus relates to a system for evaporating off high-pressure carbon dioxide botanical extraction utilizing separate heating zones and a cold finger condenser.

### BACKGROUND

Supercritical and subcritical carbon dioxide have been used since the 1930s for the extraction of natural oil products. The process was first used by the Germans for the purpose of extracting oil from shale. Since the end of World War II, a large number of CO₂extraction plants have been built for the purpose of extracting hops oil for use in beer making.

The advantages of CO₂ extraction include its ability to be tuned for different extraction parameters by adjusting temperature and pressure and the fact that it leaves no toxic solvent residues within the final product. Some of the disadvantages of CO₂ extraction are that it is a complicated process which involves a lot of technical hurdles, including pumping and pressure and temperature swings involved in the process. Another disadvantage is that current extraction methods and equipment are difficult to use and are often inefficient at evaporating the CO₂ solvent from the mixture and leaving the oil behind. If the system is under heated, the oils can freeze which can prevent efficient collection, among other issues. If the system is overheated, the extruded oils can caramelize.

The present invention seeks to remedy these issues by controlling the temperature and pressure of the oil mixture and more efficiently and completely evaporating the CO₂ from the mixture, without damaging the extracted oils.

### SUMMARY

A CO₂ extraction system which is capable of being used for extracting compounds, such as oils, from various materials including plant material and botanicals. The extraction system includes a means for supplying CO₂ to a pressure vessel. The pressure vessel is substantially comprised of two distinct portions; a top portion and a bottom portion. The top portion includes a separation zone, while the bottom portion contains a collection zone. Affixed to the bottom portion of the pressure vessel is a drain valve which is capable of allowing extracted material to exit the system.

When introducing CO₂ into the extraction system, the CO₂ may be liquefied and further saturated with additional compounds. By maintaining each of the respective top and bottom portions of the pressure vessel at differing temperatures and/or pressures, the various additional compounds may be separated from the CO₂.

### DESCRIPTION OF THE DRAWINGS

FIG. 1 is an embodiment of a CO₂ extraction system;

FIG. 2 is an embodiment of a pressure vessel used to separate CO₂;

FIG. 3 is an internal view of a pressure vessel used to separate CO₂.

### DETAILED DESCRIPTION

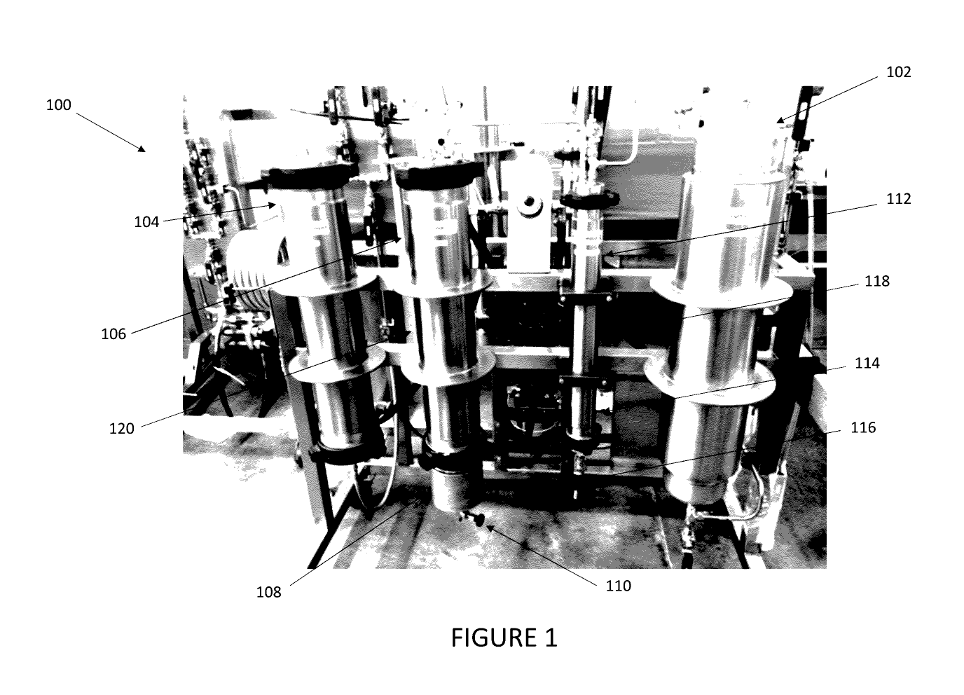

With reference to FIG. 1, shown is an embodiment of a CO₂ extraction system 100. The CO₂ extraction system 100 includes various component parts, including, but not limited to, a CO₂ accumulator 102, a pump, an extraction vessel 104, and at least one separator 106.

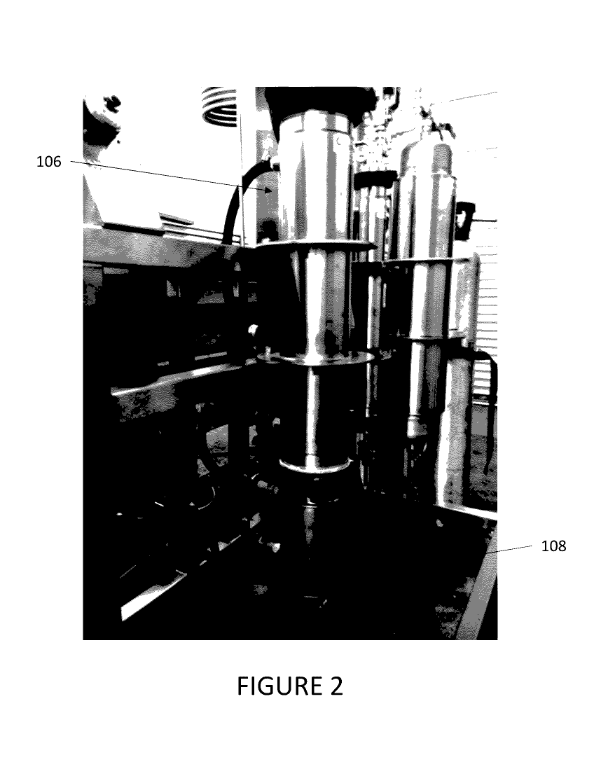

With continued reference to FIG. 1 and with reference to FIG. 2, the CO₂ extraction system 100 generally operates in the following manner. First, plant or other organic or raw materials are inserted into the extraction vessel 104. Next, liquid CO₂ is pumped from the accumulator 102, where it is stored when not in use in this process, through a conduit to the extraction vessel 104. The extraction vessel 104 is then pressurized and heated to a certain temperature so that CO₂ is compressed to a supercritical fluid upon its entry into the extraction vessel 104. In certain embodiments, the exterior of the extractor is insulated with an insulation jacket to assist in maintaining the temperature within the extractor. The liquefied CO₂ passes through the plant or other organic or raw material within the extractor and acts as a solvent, removing (i.e., extracting) various oils and compounds from the plant or other raw materials. The liquefied CO₂ containing the extracted oils and compounds is then transferred or pumped to a separator 106. The separator 106 is maintained at a different temperature and pressure than the extractor 104 which results in the separation of the oil and other extracted compounds from the liquefied CO₂ and the conversion of liquefied CO₂ into CO₂ gas. For example, in certain embodiments, the extractor 104 is maintained at a pressure of 1000 psi and the separator 106 is maintained at a pressure of 200 to about 400 psi. The oil and other extracted compounds fall into a collection vessel 108 at the bottom of the separator while the CO₂ gas is transferred or pumped through a conduit to either the CO₂ tank or to the extractor 104 to be recirculated through the system.

With continued reference to FIG. 1, the extractor 106 can have two distinct zones: a collection zone at the bottom portion of the vessel and a main body consisting of a separation zone 120 located above the collection zone. The collection zone consists of the collection vessel 108 and a release valve 110 at the base of the separator 106, which can have a separate heating unit that can allow the collection vessel 108 to maintain a different temperature than the rest of the separator 106. In certain embodiments, the collection vessel 108 can be heated to a temperature in the range of 75-100° F. while the separator 106 can be heated to a temperature in the range of 120-140° F. The temperature differential between the collection vessel 108 and the separator 106 allows for more complete separation of the volatile oils from the CO₂ solution.

With reference now to FIG. 2, an alternative view of the separator 106 may be seen. The collection vessel 108 may be further seen as comprising the bottom portion of the separator 106.

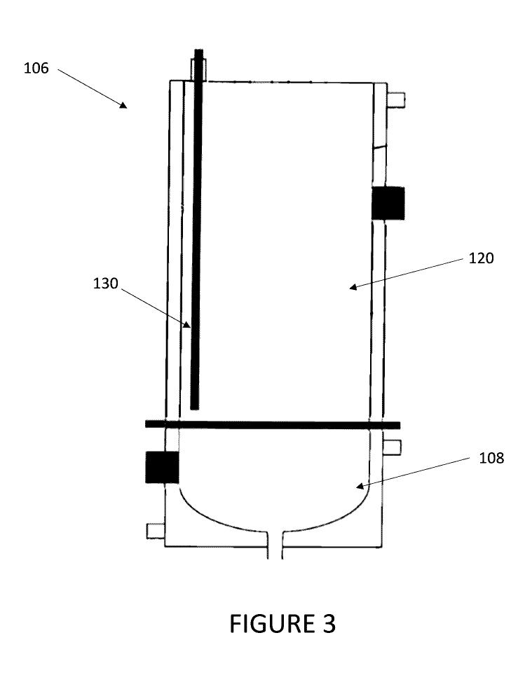

With reference now to FIG. 3, the CO₂ solution enters the collection vessel 108 at the base of the separator 106 via a bore through tube 130. The bore through tube 130 is sometimes also commonly referred to as a “dip tube.” As the supercritical CO₂ solution enters the collection vessel, the pressure is reduced which causes the CO₂ to naturally flash to a gaseous state. At the same time that the saturated liquid solution enters the separator 106 via the collection vessel 108, the heavier oils and oleoresins within the CO₂ solution will begin to sink into the collection vessel 108 where it can be collected through a release valve 110 located at the base of the collection vessel 108.

With continued reference to FIG. 3, the liquid CO₂ solution being pumped into the collection vessel 108 causes the fluid level to rise within the separator 106 until the liquid solution contacts the separation zone of the separator 106. Here, the higher temperature accelerates the matter state transition of the CO₂ from liquid to gas. As the CO₂ vaporizes, the gas rises within the separator while oils and oleoresins continue to separate and fall into the collection vessel 108.

According to one embodiment, the separator 106 which contains both the collection vessel 108 and separation zone 120 consists of a single unit. According to further embodiments, each of the collection vessel 108 and separation zone 120 are contained in separate units. When consisting of separate units, each of the collection vessel 108 and separation zone 120 may be equipped with an independent source of heat so as to maintain the respective unit at the desired temperature. The two units may further be connected to one another by any means recognized by those skilled in the art for allowing the units to maintain their intended purpose of extracting various compounds from the CO₂.

With reference to FIG. 1, in some instances it may be desired to utilize a re-boiler in connection with the separator 108. According to one embodiment, a re-boiler can be used to maximize the boiling surface which can in turn increase the speed at which the CO₂ is vaporized, thereby increasing the speed at which the extraction and separation process can take place. According to this embodiment, the re-boiler can be a second separator 112 with a separate collection zone, consisting of a second collection cup 114 and a second release valve 116, and a separate separation zone 118. In this embodiment, the second separator 112 operates much like the first separator 108 and is used to increase the boiling surface and thereby increase the separation speed.

According to another embodiment, the re-boiler can be used for gentle extractions of light and volatile mono-terpenes which require an even lower temperature at separation to maintain their integrity. This use of the re-boiler slows the extraction and separation process considerably, but minimizes the degradation and loss of these highly volatile light oils.

Embodiments disclosed herein are not necessarily in the alternative, as various embodiments may be combined or subtracted to provide the desired characteristics. Variations can be made by one having ordinary skill in the art without departing from the spirit and scope hereof. Therefore, the CO₂ extraction system should not be limited to any single embodiment, but rather construed in breadth and scope in accordance with the recitations of the appended claims. The right to claim elements and/or sub-combinations that are disclosed herein as other inventions in other patent documents is hereby unconditionally reserved.

Having thus described the invention, it is now claimed:

I claim:

1. A CO₂ extraction system comprising:
a means for supplying CO₂ to the extraction system;
a pressure vessel having a top portion and a bottom portion, wherein said top portion comprises a separation zone, said bottom portion comprises a collection zone, and said top and bottom portions are capable of being maintained at a temperature and pressure which may be independent from that of the opposing portion, and;
a drain valve affixed to said bottom portion of said pressure vessel, wherein said drain valve is capable of releasing the contents of said bottom portion from the collection zone of said pressure vessel;
wherein liquefied CO₂ is capable of being introduced into the pressure vessel via the collection zone by said means for supplying CO₂, said collection zone is capable of maintaining a pressure and temperature which allows for said liquefied CO₂ to flash to the gaseous state upon entry into said pressure vessel, said collection zone is capable of capturing any oils, compounds, or other matter or material which may be separated during the phase change, and said separation zone is capable of being maintained at a temperature and pressure different than that of said collection zone.

2. The extraction system of claim 1 wherein the means for supplying CO₂ is by way of a dip tube which enters the pressure vessel through the top portion, extends about the relative length of said pressure vessel, and releases CO₂ into the bottom portion of said pressure vessel.

3. The extraction system of claim 2 wherein the collection zone of said pressure vessel is maintained at a temperature between approximately 75° F. and approximately 100° F.

4. The extraction system of claim 3 wherein the separation zone of said pressure vessel is maintained at a temperature between approximately 120° F. and 140° F.

5. The extraction system of claim 1 wherein the top portion and bottom portion of said pressure vessel are two unique structures which are connected in such a manner so as to operate as a single vessel.

6. The extraction system of claim 5 wherein each of said top portion and bottom portion contains an independent heat source.

7. The extraction system of claim 1 wherein the spent CO₂ is removed from said pressure vessel and sent to an accumulator.

8. The extraction system of claim 1 wherein the pressure vessel has a minimum pressure rating of 1,000 psi.

9. A method of extracting compounds from an organic material comprising the following steps:
providing the extraction system of claim 1;
supplying liquefied CO₂, which contains at least one additional organic compound or material, to said collection zone of said extraction system by way of a dip tube;
heating said collection zone to a temperature between about 75° F. and about 100° F.;
heating said separation zone to a temperature between about 120° F. and about 140° F., and;
collecting the separated materials from said collection zone.
